(19) United States
(12) Patent Application Publication
(10) Pub. No.: US 2025/0289011 A1
(43) Pub. Date: Sep. 18, 2025

(54) SYSTEM AND METHOD FOR ADAPTIVE CHARGED SPRAY DEPOSITION AND FEEDBACK

(71) Applicant: SUNLESS, INC., Macedonia, OH (US)

(72) Inventors: Benjamin David Johnson, Northfield, OH (US); Yossi Har-Nov, Parkland, FL (US); Matthew R. Wancata, Cuyahoga Falls, OH (US)

(73) Assignee: SUNLESS, INC., Macedonia, OH (US)

(21) Appl. No.: 19/078,659

(22) Filed: Mar. 13, 2025

Related U.S. Application Data
(60) Provisional application No. 63/565,686, filed on Mar. 15, 2024.

(51) Int. Cl. B05B 5/053; A61M 35/00

(52) U.S. Cl. CPC B05B 5/053; A61M 35/25

(57) ABSTRACT

A system and method are provided to provide greater consistency and customization of results in charged spray implementations through improved feedback from and control of the electronic characteristics of the charging power supply. The system and method, in at least one form, use multiple levels of feedback and provide adjustments to react to the spray process.

This application claims priority to and the benefit of U.S. provisional patent application Ser. No. 63/565,686, filed on Mar. 15, 2024, the disclosure of which is incorporated herein by reference in its entirety.

### BACKGROUND

Electrically charged spray for targeted and even deposition of material is used in a variety of environments for a variety of purposes. Some examples include, without limitation, paint deposition, disinfectant application and spray tanning.

With respect to spray tanning, a key barrier to entry for spray tanning is the users' concerns about the quality and consistency of the spray application. Using a charged spray improves the end result by generating an attraction between the charged spray particles and the electrically grounded user. More complete coverage, less dependent on the user's stance in the booth, is also provided.

Currently, these known application methods provide a fixed voltage with which to charge the aerosolized spray particles such that they are attracted to the work surface. In this regard, a current accepted solution in the industry, as described, for example, in U.S. Pat. No. 7,913,938, utilizes as fixed voltage converter to step up a system level voltage (something like 5V, 12V or 24V, easily available from commercial power supplies), to a significantly higher voltage (hundreds of volts or even over 1,000V in some embodiments) at a low, safe current. This conversion is triggered by simply applying power to the system and connecting the output to the spray mechanism.

However, such current spray charging solutions are not actively adjustable, nor do they provide sufficient feedback to the system to make suitable adjustments during the spray pass. The result is that, while the small, charged spray particles may be attracted to the user as desired, such attraction is neither optimized for the variables of the current session nor actively monitored over the time period (e.g., months or years) that a spray booth is in active use.

### BRIEF DESCRIPTION

In accordance with one aspect of the presently described embodiments, an adjustable direct-current-to-direct-current (DC-DC) electrostatic power supply system is provided comprising a least one processor, at least one memory having stored thereon instructions that, when executed by the at least one processor, cause the system to: generate a voltage based on an input voltage and a control signal, apply the voltage to a spray nozzle, detect output voltage of the spray nozzle, and, control adjustment of the voltage based on the detected output between 0 and 1000 Volts.

In accordance with one aspect of the presently described embodiments, a method for adjustable direct-current-to-direct-current (DC-DC) electrostatic power supply comprises generating a voltage based on an input voltage and a control signal, applying the voltage to a spray nozzle, detecting output voltage of the spray nozzle, and, controlling adjustment of the voltage based on the detected output between 0 and 1000 Volts.

In accordance with one aspect of the presently described embodiments, a system for adaptive charged spray deposition and feedback having a spray nozzle and an input voltage applied thereto is provided comprising at least one processor, at least one memory having stored thereon instructions that, when executed by the at least one processor, cause the system to adjust the input voltage, generate alternating voltage based on the input voltage and a control signal, transform the alternating voltage to alternating high voltage, rectify the alternating high voltage to a high voltage direct current output, apply the high voltage direct current output to the spray nozzle, detect or measure the high voltage direct current output, and, control adjustment of the input voltage based on the detected or measured high voltage direct current output.

In accordance with another aspect of the present exemplary embodiment, the system control signal is a pulse width modulated signal.

In accordance with another aspect of the present exemplary embodiment, the system control signal is an oscillator signal.

In accordance with another aspect of the present exemplary embodiment, the high voltage direct current output is measured based on initiation of the feedback session, reading of processor settings and enablement of pulse width modulation and voltage functions.

In accordance with another aspect of the present exemplary embodiment, the system is further caused to control adjustment of the input voltage based on sensor feedback.

In accordance with another aspect of the present exemplary embodiment, the system sensors are read or measured based on initiation of the feedback session, reading of processor settings and enablement of pulse width modulation and voltage functions.

In accordance with one aspect of the presently described embodiments, a system for adaptive charged spray deposition and feedback having a spray nozzle and an input voltage applied thereto, comprises a least one processor, at least one memory having stored thereon instructions that, when executed by the at least one processor, cause the system to: adjust the input voltage, generate alternating voltage based on the input voltage and a pulse width modulation signal, transform the alternating voltage to alternating high voltage, rectify the alternating high voltage to a high voltage direct current output, apply the high voltage direct current output to the spray nozzle, detect or measure the high voltage direct current output, and, control adjustment of the input voltage based on the detected or measured high voltage direct current output.

In accordance with another aspect of the present exemplary embodiment, the system high voltage direct current output is measured based on initiation of the feedback session, reading of processor settings ad enablement of pulse width modulation and voltage functions.

In accordance with another aspect of the present exemplary embodiment, the system is further caused to control adjustment of the input voltage based on sensor feedback.

In accordance with another aspect of the present exemplary embodiment, the system sensors are read or measured based on initiation of the feedback session, reading of processor settings and enablement of pulse width modulation and voltage functions.

In accordance with one aspect of the presently described embodiments, a system for adaptive charged spray deposition and feedback having a spray nozzle and an input voltage applied thereto, comprises a least one processor, at least one memory having stored thereon instructions that, when executed by the at least one processor, cause the system to: adjust the input voltage, generate alternating voltage based on the input voltage and an oscillator signal, transform the alternating voltage to alternating high voltage, rectify the alternating high voltage to a high voltage direct current output, apply the high voltage direct current output to the spray nozzle, detect or measure the high voltage direct current output, and, control adjustment of the input voltage based on the detected or measured high voltage direct current output.

In accordance with another aspect of the present exemplary embodiment, the system high voltage direct current output is measured based on initiation of the feedback session, reading of processor settings ad enablement of pulse width modulation and voltage functions.

In accordance with another aspect of the present exemplary embodiment, the system is further caused to control adjustment of the input voltage based on sensor feedback.

In accordance with another aspect of the present exemplary embodiment, the system sensors are read or measured based on initiation of the feedback session, reading of processor settings and enablement of pulse width modulation and voltage functions.

In accordance with one aspect of the presently described embodiments, a method for adaptive charged spray deposition and feedback in a system having a spray nozzle and an input voltage applied thereto, comprises: adjusting the input voltage, generating alternating voltage based on the input voltage and a control signal, transforming the alternating voltage to alternating high voltage, rectifying the alternating high voltage to a high voltage direct current output, applying the high voltage direct current output to the spray nozzle, detecting or measuring the high voltage direct current output, and, controlling adjustment of the input voltage based on the detected or measured high voltage direct current output.

In accordance with one aspect of the presently described embodiments, the method further comprises controlling adjustment of the input voltage based on sensor feedback.

In accordance with one aspect of the presently described embodiments, the method control signal is a pulse width modulated signal.

In accordance with one aspect of the presently described embodiments, the method control signal is an oscillator signal.

### BRIEF DESCRIPTION OF THE DRAWINGS

FIG. 1 is a representative view of an example spray nozzle to which the presently described embodiments may applied;

FIG. 2 is a block diagram of a system according to the presently described embodiments;

FIGS. 3A and 3B illustrate flowcharts for feedback methods according to the presently described embodiments; and,

FIG. 4 illustrates an example flowchart for a method according to the presently described embodiments.

### DETAILED DESCRIPTION

The present exemplary embodiments provide greater consistency and customization of results in charged spray implementations through improved feedback from and control of the electronic characteristics of the charging power supply. The present exemplary embodiments, in at least one form, use multiple levels of feedback and provide adjustments to react to the spray process. In at least some forms, the adjustments are iterative, ongoing and may be immediate or near immediate in time.

The present exemplary embodiments relate, for example, to charged spray applications including those mentioned above. They find particular application in conjunction with spray tanning and will be described with particular reference thereto. One example implementation of the presently described embodiments is found as part of an automated spray booth that utilizes custom spray nozzles to supply a metered dose of skincare treatment solution to the user. Another example implementation of the presently described embodiments is for hand-held spray guns that are utilized by trained technicians to apply bespoke skin treatment results to the customer. However, it is to be appreciated that the present exemplary embodiments are also amenable to other like applications in spray tan environments as well as other applications using charged spray processes.

More particularly, the present exemplary embodiments, in at least one form, implement a new electrostatic power supply design, as well as additional feedback paths to monitor and adjust the performance of the electrostatic spray over the course of a given treatment.

In one form, an adjustable direct-current-to-direct-current (DC-DC) electrostatic power supply system having capability between 0 and 1000 Volts is provided. As an example, the system comprises a least one processor, at least one memory having stored thereon instructions that, when executed by the at least one processor, cause the system to generate a voltage based on an input voltage and a control signal, apply the voltage to a spray nozzle, detect output voltage of the spray nozzle, and control adjustment of the voltage based on the detected output between 0 and 1000 Volts. As one further example, the control signal is a pulse width modulation signal. As another example, the control signal is a oscillator signal.

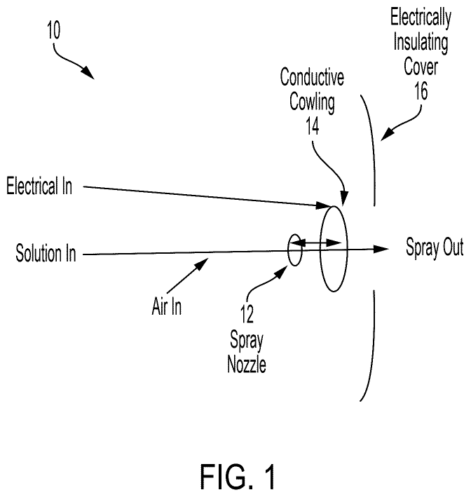

With reference to FIG. 1, an example spray nozzle to which the presently described embodiments may be applied is illustrated in representative form. As shown, a spray nozzle configuration 10 includes a spray nozzle 12, having a fluid channel terminating in a fluid tip (not shown) in most embodiments. The spray nozzle 12 is surrounded by a conductive cowling or an electrode 14. The configuration is also provided with an electrically insulative cover 16. The nozzle configuration of FIG. 1 may take a variety of forms, including the form(s) of the nozzle configuration(s) shown in U.S. Pat. No. 7,913,938, which is incorporated herein by reference in its entirety (“the '938 patent”). The operation of its disclosed nozzle configurations as charged spray nozzles is also described in the '938 patent.

In general, however, as representatively shown in FIG. 1, charged spray nozzles are supplied with solution or fluid to be sprayed and air that is added to the fluid stream as it progresses through the fluid channel to the fluid tip. The electrode 14 is charged. As such, as spray fluid exits the spray nozzle and travels through the electrode, it is charged to a desired voltage. It should be appreciated that the distance from the spray nozzle to the conductive cowling that charges the spray is a factor, e.g., a critical or substantial factor, in improving, e.g., maximizing or optimizing, the charge applied to the aerosolized spray particles. As noted above, using a charged spray configuration improves the end result by generating an attraction between the charge spray particles and the electrically grounded user.

In the operation of the system according to the presently described embodiments which include the spray nozzle configuration of FIG. 1 or another suitable charged spray nozzle system or configuration, similar to existing solutions, the present exemplary embodiments use a standard low voltage (e.g., 12V in this case) that is supplied to the spray system and/or component. This voltage is then stepped up to a higher active voltage to charge the spray. In the present exemplary embodiments, however, the exact voltage that reaches the sprayer is adjustable to allow for control of the output voltage. In addition, a pulse width modulation (PWM) circuit is included for even finer control of the output. The PWM circuit facilitates the modulation of the incoming voltage to smooth and control the output. In combination, the voltage and PWM control provide increased, e.g., infinite, adjustability of the output voltage and, thus, the charge of the spray. As an alternative to the PWM circuit, an oscillator circuit may be implemented.

The presently described embodiments also include an analog to digital (ADC) feedback line that directly reports the output voltage of the system back to the control unit. This allows for direct monitoring of the performance of the system. This can result in more repeatable spray sessions by ensuring that, regardless of other systemic or environmental factors, the same voltage is being applied to the spray for every session. Or, even more dynamically, the output voltage can be changed actively during the spray to provide other results improvements.

In addition, the present exemplary embodiments, in at least one form, implement additional levels of feedback to provide further data to refine the spray characteristics for the best possible spray deposition result. At one level, the electrical or environmental characteristics within the spray chamber could be monitored and the output voltage varied based on that data. This could include feedback about the chamber's temperature or humidity, a measurement of the spray density in the chamber, or a current reading from the user's grounding footpads to measure the deposition of solution/electrical charge during the session. At another level, the actual deposition results on the user could be monitored and the output voltage or spray pattern varied based on that data. This could include feedback about changes in the user's external coverage via a camera or other optical sensing method or a remote measurement of the user's skin moisture to measure saturation due to spray.

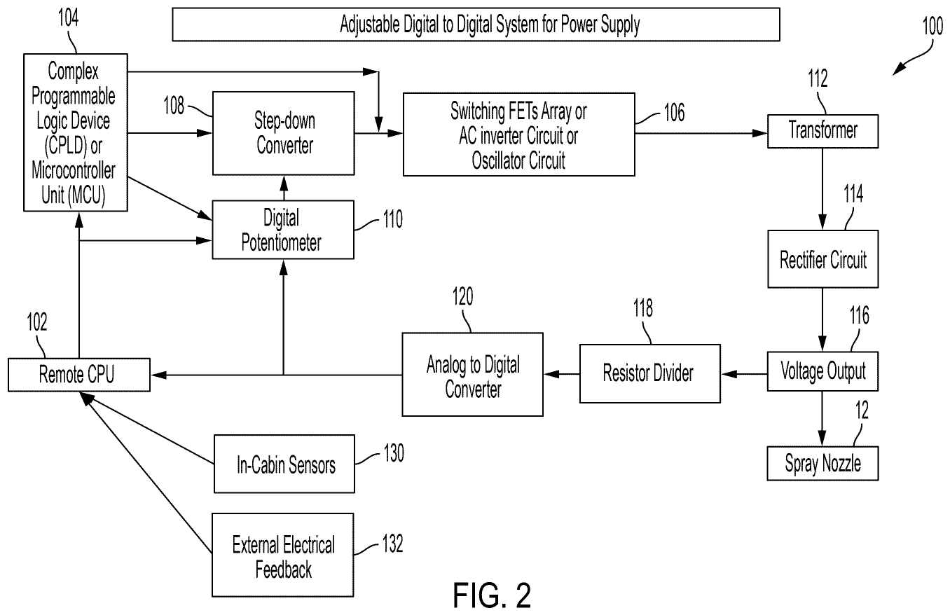

With reference to FIG. 2, a block diagram of a system 100 according to the presently described embodiments is illustrated. As shown, the system 100, in at least one form, is implemented as an Adjustable Digital to Digital Power Supply system, in a range of, e.g., +10V to +1800V, that includes suitable feedback and control elements to achieve the functionality of the presently described embodiments. The system 100 includes a Remote CPU 102 (or central processing unit) that functions as a host computer configured to control, evaluate, and dynamically adjust the circuit parameters to set the output voltage as desired. The Remote CPU 102 can also drive the feedback loop (to be described below). A Complex Programmable Logic Device (CPLD) or Microcontroller Unit (MCU) 104 is also provided. The CPLD/MCU 104 manages the communication with the remote CPU. Based on its internal defaults and communication with the Remote CPU 102, this device generates a precise clock and adjusts, for example, in one form, the pulse width modulated (PWM) outputs that control the switching FETs array (or AC Inverter circuit) 106. Also, the element 106 may take the form of an oscillator circuit (to be described hereinafter), as an alternative to pulse width modulation. Of course, it will be appreciated that the elements 102 and/or 104 will be configured to accommodate the implementation of the oscillator circuit.

As shown, it will be appreciated that the Remote CPU 102 and CPLD/MCU 104 each have at least one processor and at least one memory. Of course, the at least one processor is configured to execute code, instructions or routines that may be stored on the at least one memory (or on other appropriate memories) to trigger or cause components of the system 100 (including, but not limited to, the Remote CPU 102, CPLD/MCU 104 and/or memory elements or other elements) to perform or function in suitable manners to implement the presently described embodiments and other objectives.

It will be appreciated by those of skill in the art, upon a reading of the present specification, that the Remote CPU 102 and CPLD/MCU 104 as described, as well as the described processors and memories, may take a variety of forms to implement the presently described embodiments. The processors can be embodied in a variety of hardware forms, such as digital processors, single-core processors, multi-core processors, or coprocessors, or the like. The memories may be any type of tangible non-transitory computer readable medium such as random-access memory (RAM), read only memory (ROM), magnetic disk or tape, optical disk, flash memory, or holographic memory, or the like. In at least one embodiment, the memories may comprise a combination of random-access memory and read only memory portions.

Referring back to FIG. 2, in the example shown, disposed between the CPLD/MCU 104 and the element 106 is a Step-down Converter 108. The converter 108 generates a stepped-down voltage source which is lower than its input voltage. The exact voltage output by this device is directly proportional to the voltage on its feedback pin. The voltage on the feedback pin is controlled by the Digital Potentiometer 110. The Digital Potentiometer 110 adjusts the resistance between two pins based on a setting provided either by the Remote CPU 102 or the CPLD/MCU 104. This resistance value controls the output voltage of the step-down converter 108 above. It will be appreciated that the range of output voltage for the step-down converter could be within any suitable range. One example is a step-down converter have a range of −0.3V to 28V, with the upper limit being dependent on the input voltage. Another example step down converter has an output range of +4V to +10V.

As noted, a Switching FETs Array or AC inverter or Oscillator circuit 106 is provided. In at least one form, the inputs to this circuit 106 are the output voltage from the step-down converter 108 and the PWM signal from the CPLD/MCU 104. The output could vary but, in this example, the output is an alternating (zero-crossing) voltage with a frequency equal to two (2) times the PWM signal frequency. This circuit can also have a set frequency determined by the passive components in the AC inverter circuit.

In another embodiment, the element 106 takes the form of an oscillator circuit. Although the configurations for an oscillator circuit may vary to achieve the objectives of the presently described embodiments, in one example, a Baxandall Oscillator (also known as a resonant-Royer oscillator) is implemented. A Baxandall Oscillator is a self-resonating oscillator that converts a DC input into an AC output with the help of a feedback coil on the primary side of a transformer. The transformer has a center tapped primary, into which the DC voltage is supplied. The positive half of the sine wave is generated from a high-side transistor whose gate current is initially supplied by the DC source. This causes current to flow from the center tap through the high-side winding of the primary. The negative half of the sine wave is then supplied by a low-side transistor whose gate current lags the high-side's transistor due it's connection through the feedback winding of the transformer. When the low-side transformer begins to conduct, the current opposes the high-side and forces current to flow in the opposite direction. This then causes the high-side base current to increase relative to the low-side's base current until the primary coil's current has flipped high again. This oscillation continues until the DC supply is removed. The frequency at which the oscillator functions is controlled and tuned by the LC relationship between the transformer's primary coil inductance and a capacitor which spans the transformer's primary coil. The output voltage is increased relative to the winding ratio between the primary and secondary coils of the inductor.

With continuing reference to FIG. 2, a step-up transformer 112 is implemented. In one form, the step-up transformer includes a large secondary-to-primary ratio (e.g., 100:1) used to generate alternating high-voltage. The output of the transformer 112 is provided to Rectifier Circuit 114—in which a collection of high-voltage diodes arranged in a half- or full-bridge rectifier circuit with a smoothing capacitor achieve a high-voltage DC output (shown as Voltage Output 116). The Output Voltage 116 is provided to the spray nozzle 12.

According to the presently described embodiments, a suitable feedback path is also provided. Feedback according to the presently described embodiments will facilitate active adjustment of the spraying solution or fluid by providing information to the system to make suitable adjustments during the spray pass. The result is that charged spray particles are attracted to the user as desired and such attraction is improved, e.g., optimized, for the variables of the current session. Also, performance is actively monitored over a desired time period (e.g., months or years) that a spray booth is in active use.

The feedback path or loop may take a variety of forms. The feedback loop can be driven in any suitable manner; however, as shown, the Remote CPU 102 can drive the feedback loop and/or the onboard CPLD chip 104 can directly drive the feedback loop.

In one form of the feedback path or loop, a Resistor Divider 118 is implemented. Since the analog to digital converter (ADC) 120 can only detect low DC voltages, this resistor divider circuit brings the output voltage level down to a signal-level DC voltage (e.g., Max 3.3V, 5V). As noted, the Analog to Digital Converter 120 is provided. The input signal to this device is the output voltage 116 of the charged spray system-which has been scaled down by the resistor divider. The output can be evaluated by the Remote CPU 102 or CPLD/MCU 104 for data collection and further adjustment to circuit parameters through, for example, the Digital Potentiometer 110 or the PWM settings for the PWM signal delivered from the CPLD/MCU 104.

In operation, in one example, a suitable, e.g., an ideal, voltage of, for example, 1000V typically provides the best charging to the aerosolized spray. It is known that passing the spray through the conductive cowling reduces the measured output voltage (e.g., this is a very useful measure for knowing when the spray is active) and the internal feedback can be used to ramp the PWM value up to return the measured output voltage to 1000V during the spray.

Also shown in FIG. 2 are other inputs to the Remote CPU 102. For example, the Remote CPU 102 may receive data from in-cabin sensors 130 or other external electrical feedback 132. External sensors could include environmental parameters, electrical feedback, color or visual information (via camera or sensor), spray density measurements or other defining spray quality measures.

In at least one form, a sensor(s) 130 in the cabin reads the temperature and humidity of the environment around the tanner. In less humid conditions before the spray, the input voltage and PWM values can be modified to result in a greater output voltage value to take advantage of the environmental conditions. The output voltage 116 can then be ramped down as the spray continues and increases the humidity in the cabin.

In at least one form, an additional electric circuit, such as circuit(s) 132, measures the additional charge applied through the tanner to the grounding plates in the booth. The output voltage of the circuit is modulated to maintain, for example, a consistent 10 uA of current through this measurement circuit.

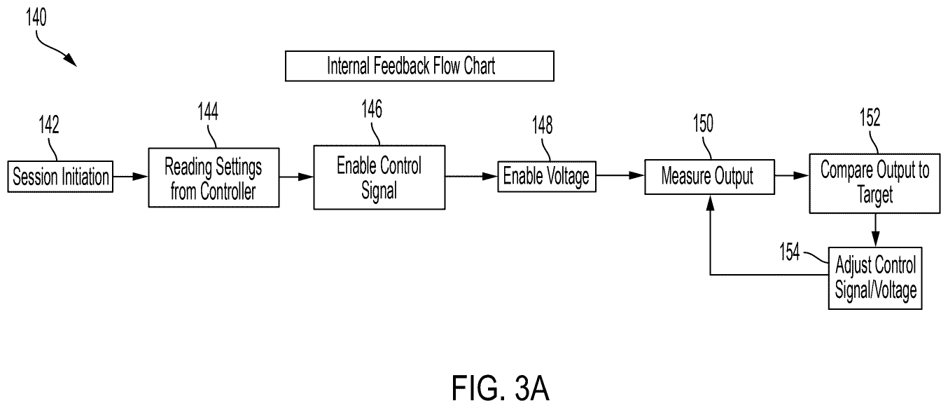

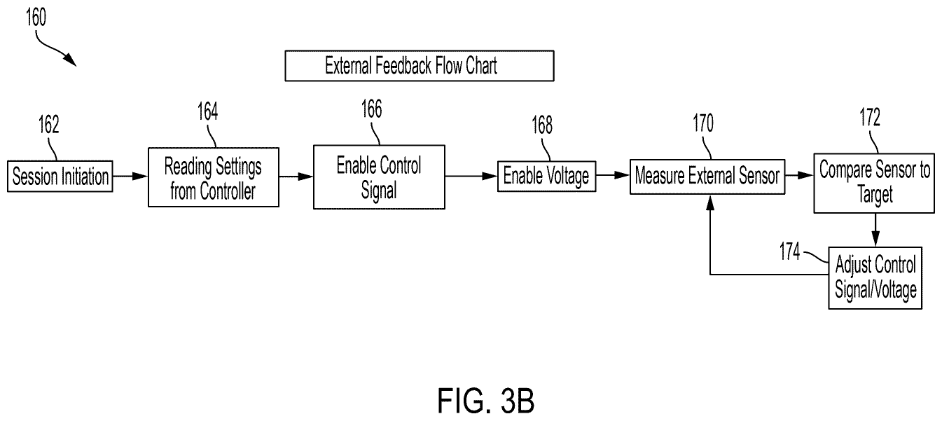

With reference to FIG. 3A, a feedback flowchart illustrating an example internal feedback method 140 according to the presently described embodiments is shown. As noted, the feedback loop can be driven in any suitable manner; however, as shown, the Remote CPU 102 can drive the feedback loop and/or the onboard CPLD chip 104 can directly drive the feedback loop.

In one example, a session is initiated (at 142) and settings of the controller (e.g., one or both of Remote CPU 102 or CPLD/MCU 104) are read (at 144). Control signals, e.g., PWM or oscillator signals, and voltage functions are enabled (at 146, 148). The output voltage 116 is then measured (at 150). The measured value(s) is compared to a target value (at 152). It should be appreciated that the target value for voltage could be a static value or could vary based on a pre-defined time-value chart based on experimental results. It should also be appreciated that any suitable approach will suffice. The Control Signal/voltage is then adjusted (at 154).

With reference to FIG. 3B, a feedback flowchart illustrating an example external feedback method 160 according to the presently described embodiments is shown. As noted, the feedback loop can be driven in any suitable manner; however, as shown, the Remote CPU 102 can drive the feedback loop and/or the onboard CPLD chip 104 can directly drive the feedback loop.

In one example, a session is initiated (at 162) and settings of the controller (e.g., one or both of Remote CPU 102 or CPLD/MCU 104) are read (at 164). Control signals, e.g., PWM or oscillator signals, and voltage functions are enabled (at 166, 168). The external sensors, such as those represented at 130, 132 of FIG. 2, are then measured (at 170). The measured value(s) is compared to a target value (at 172). It should be appreciated that the target value for sensor values could be a static value or could vary based on a pre-defined time-value chart based on experimental results. It should also be appreciated that any suitable approach will suffice. The Control Signal/voltage is then adjusted (at 174).

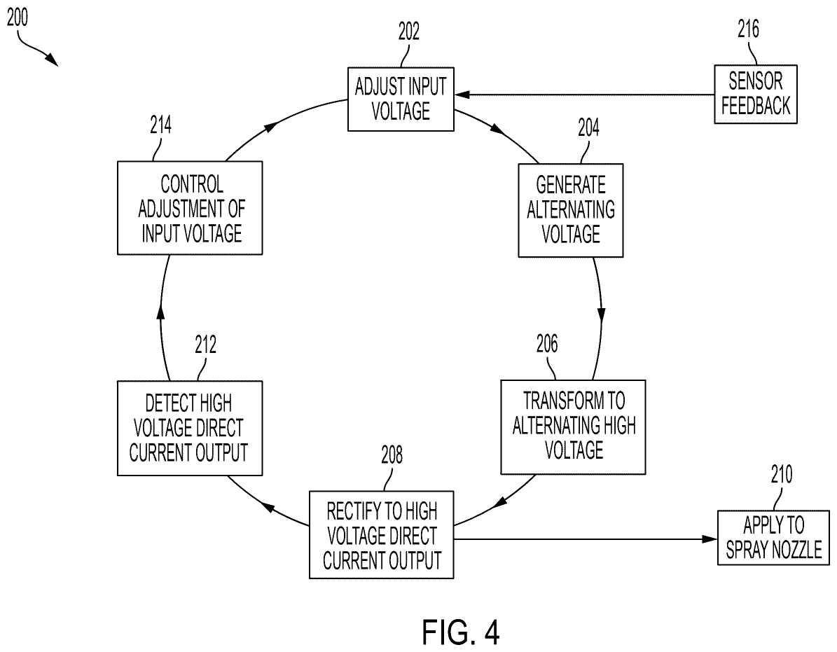

With reference to FIG. 4, an example method 200 according to the presently described embodiments is shown. In this regard, it should be appreciated that the system implementing this or other methods according to the presently described embodiments may take a variety of forms. As noted above, one example implementation of the presently described embodiments is found as part of an automated spray booth that utilizes custom spray nozzles to supply a metered dose of skincare treatment solution to the user. Another example implementation of the presently described embodiments is for hand-held spray guns that are utilized by trained technicians to apply bespoke skin treatment results to the customer. Other like applications in spray tan environments, as well as other applications using charged spray processes, may also provide implementations for the presently described embodiments. In any of these systems to which the example method 200 may be applied, an initial input voltage is provided to the system. This initial input voltage could be provided in any number of manners.

With respect to method 200, the initial voltage supplied to the system is adjusted (at 202) to generate an adjusted input voltage. It should be appreciated that such adjustment, according to at least one form of the presently described embodiments, may comprise a step-down of voltage and/or adjustment based on the feedback pins, as noted in connection with the description of FIG. 2. An alternating voltage based on the adjusted input voltage and a control signal, e.g., a pulse width modulated (PWM) or oscillating signal, is generated (at 204). The alternating voltage is transformed to an alternating high voltage (at 206). As described above, in one form, this could be accomplished using a suitable step-up transformer. A high voltage direct current output is generated by rectifying the alternating high voltage (at 208). This high voltage direct current output is applied to the spray nozzle (at 210) for emission to the intended target, e.g., the skin of a spray tan user.

According to the presently described embodiments, additional functionality is provided. In this regard, the high voltage direct current output is detected and/or measured (at 212). Adjustment of the high voltage direct current output is implemented, as necessary (at 214). It should also be appreciated that the sensor feedback, examples of which are discussed herein, also contribute, as appropriate or desired, to the adjustment of the high voltage direct current output (at 216). As illustrated, in at least one example of the presently described embodiments, the method 200 is an on-going and iterative process to provide improved performance of the charged spray system to which it is applied.

As noted, such feedback according to the presently described embodiments will facilitate active adjustment of the spraying solution or fluid by providing information to the system to make suitable adjustments during the spray pass. The result is that charged spray particles are attracted to the user as desired and such attraction is improved, e.g., optimized, for the variables of the current session. Also, performance is actively monitored over a desired time period (e.g., months or years) that a spray booth is in active use.

It should be appreciated that this method 200 may be implemented using systems described in connection with FIGS. 1 and 2 or any other suitable system. It should also be recognized that the method 200 and other methods and techniques according to the presently described embodiments (including the methods of FIG. 3(a) and FIG. 3(b)) may be implemented using a variety of different configurations of hardware, software code or instructions (e.g., stored on appropriate non-volatile memory devices and/or non-transitory computer-readable medium and executed by suitable processors) and other functional components. For example, various hardware configurations and software routines may be used to trigger or cause the various components of the system to perform necessary functions to achieve the objectives of the presently described embodiments.

Overall, advantages of the presently described embodiments include:
More consistent and repeatable spray deposition
Assurance that more solution ends up on the user (less waste)
Valuable feedback data for both service and refinement

Skin treatment spray deposition has historically been a straightforward application. Many current implementations do not even use electrostatic spray mechanisms. Heretofore, purely mechanical systems have provided a sufficient solution in most cases. However, as technology improves and users expect better and more consistent results, implementation of monitoring, feedback, and iterative/ongoing adjustment has become more valuable and necessary. Simple, fixed voltage conversion has existed for some time. Indeed, current implementations utilize CFL light bulb ballasts adapted for this application to convert the voltage. But, by applying digital adjustments to this otherwise analog process and pairing the digital voltage conversion to both internal and external feedback mechanisms, the present exemplary embodiments provide superior results in the same form factor.

The presently described embodiments have been described with reference to various examples for implementation. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. An adjustable direct-current-to-direct-current (DC-DC) electrostatic power supply system comprising:
a least one processor;
at least one memory having stored thereon instructions that, when executed by the at least one processor, cause the system to:
generate a voltage based on an input voltage and a control signal;
apply the voltage to a spray nozzle;
detect output voltage of the spray nozzle; and,
control adjustment of the voltage based on the detected output between 0 and 1000 Volts.

2. A method for adjustable direct-current-to-direct-current (DC-DC) electrostatic power supply, the method comprising:
generating a voltage based on an input voltage and a control signal;
applying the voltage to a spray nozzle;
detecting output voltage of the spray nozzle; and,
controlling adjustment of the voltage based on the detected output between 0 and 1000 Volts.

3. A system for adaptive charged spray deposition and feedback having a spray nozzle and an input voltage applied thereto, the system comprising:
at least one processor;
at least one memory having stored thereon instructions that, when executed by the at least one processor, cause the system to:
adjust the input voltage;
generate alternating voltage based on the input voltage and a control signal;
transform the alternating voltage to alternating high voltage;
rectify the alternating high voltage to a high voltage direct current output;
apply the high voltage direct current output to the spray nozzle;
detect or measure the high voltage direct current output; and,
control adjustment of the input voltage based on the detected or measured high voltage direct current output.

4. The system as set forth in claim 3, wherein the control signal is a pulse width modulated signal.

5. The system as set forth in claim 3, wherein the control signal is an oscillator signal.

6. The system as set forth in claim 3, wherein the high voltage direct current output is measured based on initiation of the feedback session, reading of processor settings and enablement of pulse width modulation and voltage functions.

7. The system as set forth in claim 3, wherein the system is further caused to control adjustment of the input voltage based on sensor feedback.

8. The system as set forth in claim 7, wherein sensors are read or measured based on initiation of the feedback session, reading of processor settings and enablement of pulse width modulation and voltage functions.

9. A system for adaptive charged spray deposition and feedback having a spray nozzle and an input voltage applied thereto, the system comprising:
a least one processor;
at least one memory having stored thereon instructions that, when executed by the at least one processor, cause the system to:
adjust the input voltage;
generate alternating voltage based on the input voltage and a pulse width modulation signal;
transform the alternating voltage to alternating high voltage;
rectify the alternating high voltage to a high voltage direct current output;
apply the high voltage direct current output to the spray nozzle;
detect or measure the high voltage direct current output; and,
control adjustment of the input voltage based on the detected or measured high voltage direct current output.

10. The system as set forth in claim 9, wherein the high voltage direct current output is measured based on initiation of the feedback session, reading of processor settings ad enablement of pulse width modulation and voltage functions.

11. The system as set forth in claim 9, wherein the system is further caused to control adjustment of the input voltage based on sensor feedback.

12. The system as set forth in claim 11, wherein sensors are read or measured based on initiation of the feedback session, reading of processor settings and enablement of pulse width modulation and voltage functions.

13. A system for adaptive charged spray deposition and feedback having a spray nozzle and an input voltage applied thereto, the system comprising:
a least one processor;
at least one memory having stored thereon instructions that, when executed by the at least one processor, cause the system to:
adjust the input voltage;
generate alternating voltage based on the input voltage and an oscillator signal;
transform the alternating voltage to alternating high voltage;
rectify the alternating high voltage to a high voltage direct current output;
apply the high voltage direct current output to the spray nozzle;
detect or measure the high voltage direct current output; and,
control adjustment of the input voltage based on the detected or measured high voltage direct current output.

14. The system as set forth in claim 13, wherein the high voltage direct current output is measured based on initiation of the feedback session, reading of processor settings ad enablement of pulse width modulation and voltage functions.

15. The system as set forth in claim 13, wherein the system is further caused to control adjustment of the input voltage based on sensor feedback.

16. The system as set forth in claim 15, wherein sensors are read or measured based on initiation of the feedback session, reading of processor settings and enablement of pulse width modulation and voltage functions.

17. A method for adaptive charged spray deposition and feedback in a system having a spray nozzle and an input voltage applied thereto, the method comprising:
adjusting the input voltage;
generating alternating voltage based on the input voltage and a control signal;
transforming the alternating voltage to alternating high voltage;
rectifying the alternating high voltage to a high voltage direct current output;
applying the high voltage direct current output to the spray nozzle;
detecting or measuring the high voltage direct current output; and,
controlling adjustment of the input voltage based on the detected or measured high voltage direct current output.

18. The method as set forth in claim 17, further comprising controlling adjustment of the input voltage based on sensor feedback.

19. The method as set forth in claim 17, wherein the control signal is a pulse width modulated signal.

20. The method as set forth in claim 17, wherein the control signal is an oscillator signal.
